(19) United States
(12) Patent Application Publication
(10) Pub. No.: US 2014/0099360 A1
(43) Pub. Date: Apr. 10, 2014

(54) Gas Storage Vessel, Hydrogen-Charging Method, and Hydrogen-Charging Apparatus

(71) Applicant: Keon Hwan Cho, Seoul (KR)

(72) Inventor: Keon Hwan Cho, Seoul (KR)

(73) Assignee: Do Yoon Kim, Seoul (KR)

(21) Appl. No.: 13/648,485

(22) Filed: Oct. 10, 2012

(51) Int. Cl. A61K 9/48; B29C 41/12

(52) U.S. Cl. 424/451; 264/279

(57) ABSTRACT

The present invention relates to a technique for delivering gases into the human body or generating gases inside the human body, such as a method for encapsulating gases and a gas storage vessel. The present invention relates to a gas storage capsule and to a method for producing the gas storage capsule, wherein the capsule is dissolved in water such that the air-tightness thereof is gradually degraded and the gas contained in the capsule can be gradually leaked to the outside of the capsule. The present invention also relates to a hydrogen-generating material which directly generates hydrogen gases in vivo, and to a hydrogen-generating candy for directly encapsulating hydrogen using the material and delivering the thus-encapsulated hydrogen into the intestines, as well as to a method for producing the candy.

### REFERENCE TO RELATED APPLICATIONS

This is a continuation of pending International Patent Application PCT/KR2011/002536 filed on Apr. 11, 2011, which designates the United States and claims priority of Korean Patent Application No. 10-2010-0034945 filed on Apr. 15, 2010, and Korean Patent Application No. 10-2010-0127087 filed on Dec. 13, 2010, the entire contents of which are incorporated herein by reference.

### FIELD OF THE INVENTION

The present invention relates to a gas storage vessel and method and apparatus for charging hydrogen, and more particularly, to a technology, such as a hydrogen-charged vessel, a hydrogen-generating candy, and so on, for directly providing gases into the human body or generating gases inside the human body.

### BACKGROUND OF THE INVENTION

Capsules for storing and packing oral wholesome foods or medicines have been widely used, and are divided into hard capsules and soft capsules according to materials of the capsules, and may be also used to store powder or liquid. The capsules may not be in an airtight condition according to contents stored in the capsules.

As the vessels for packing oral wholesome foods, medicines, or favorite foods, hard candies are sometimes used. Korean Patent No. 0163252 discloses a method of producing sugar-free hard candies in which hydrogen-contained saccharide is used in carbohydrate compound (syrup) in order to be in stable condition and help micro-crystallization (crystallization of crystalline polyols) in the case of a high water content.

Korean Patent Laid-open No. 10-2009-009794 discloses a capsule candy consisting of: a candy body which is empty at the center; a capsule accommodated in the empty space of the candy body and having a water-soluble film; and various contents (liquid, powder, or solid) contained in the film of the capsule.

The soft capsule is produced through the steps of: preparing chargeable contents by mixing main ingredients with diluting agent or through solubilization using a surfactant; making the film which has a thickness of 0.60 to 0.80 mm when the contents are charged; and being molded into the soft capsule according to a molding process. The drug delivery system that the capsule is put into a user's mouth and absorbed in the stomach or the intestine is generally used is generally used.

The soft capsule is mainly made of gelatin and is generally made through the steps of: putting melted gelatin into a spreader box; spreading and coating melted gelatin into a sheet form on a rotary drum roller in the spreader box to a wanted thickness; passing the gelatin sheet through a pair of die roller to such that the gelatin sheet is molded into a capsule.

Korean Patent No. 10-0647034 published on Nov. 23, 2006 discloses a soft capsule and a method of manufacturing the same, and Korean Patent No. 20-0445951 published on Sep. 11, 2009 discloses an apparatus for manufacturing soft capsules. The apparatus for manufacturing soft capsules includes a sheet molding unit having a spreader box and a cooling drum; a capsule molding unit having die rollers and a crude liquid discharging nozzle; and a capsule recovering unit for recovering the molded capsule. The soft gelatin capsule is manufactured into a capsule through the steps of: putting melted gelatin into the spreader box; spreading the melted gelatin in a sheet form on the rotary drum roller to a wanted thickness; passing the gelatin sheet through a pair of the die rollers; and cutting and bonding the gelatin sheet while injecting contents through the crude liquid discharging nozzle.

Such a soft capsule according to the prior art has been mainly used as a vessel for packing solid or liquid contents, and allows users to easily take the packed solid powder or liquid (for instance, Omega 3).

In relation with health of the human body, nowadays, people focus on the role of hydrogen. It is widely known that excessive active oxygen is harmful to the human body and it has been found that hydrogen-rich water is highly effective in prevention and treatment of adult diseases, such as cancer or diabetes, and hence, recently, many studies on hydrogen are proceeding actively at home and abroad. Moreover, the theory that hydrogen-rich water is effective in restraining oxidation of active oxygen by removing the active oxygen which is the cause of various diseases recently gets a lot of attention in the medical world.

Hydrogen gas, carbon dioxide, and methane gas containing mixed gas are generated while food is delivered to the large intestine without being hydrolyzed in the small intestine and is fermented as prey to colon bacteria flora due to enzymes which are not found in the digestive system or lacking enzymes, and the fact is known through various experiments and reports released under the name of fart and breath hydrogen. The experiments and reports are mainly studies on inconvenience in life due to excessive fart, on biorhythm and generation of gas, on pathophysiological symptoms such as celiac diseases, and on restrain of gas generation inside intestine to improve or cure the diseases and methods for reducing such symptoms or diseases.

Since the theory that hydrogen molecules are delivered to the inside of cells along the respiratory organ, the digestive organs, and various blood vessels and show effective anti-oxidant action has been recently introduced, the biochemical preventive method utilizing edible plants, which is a part of food chemistry, has been studied, and so, many reports and study presentations on effects of hydrogen molecules generated inside the intestine are gradually increasing in Japan, China, United States, and so on through various animal tests and clinical demonstrations. Particularly, the scholarship meeting of Hydrogen Research Institute in Japan handles reports on effects of hydrogen molecules generated by colon bacteria flora. Furthermore, some of antidiabetic agents or obesity medicine serve to prevent carbohydrate from being absorbed at the small intestine, and hence, lots of gases containing hydrogen gases are formed in the large intestine.

Korean Patent No. 10-0686945 discloses an apparatus for making hydrogen-rich drinking water, which has a hydrogen purifier filter structure generating hydrogen purifying water by mounting a hydrogen purifier using hydrogen purifying catalyst to an existing water purifier using electrolysis.

Korean Patent No. 10-0552003 discloses a cold and warm water purifier which provides hydrogen-rich water to generate hydrogen by mounting a bubble generator and a ceramic rod to the existing water purifier.

Korean Patent No. 10-0529006 discloses a method of generating hydrogen-rich water and a hydrogen-rich water generator, which can convert drinking water into hydrogen-rich water by generating hydrogen gas through reaction between drinking water contained in a plastic bottle and magnesium particles and purify drinking water by silver particles.

However, the hydrogen purifier and the bubble generator using electrolysis have several problems in that they cannot be used in places where a water purifier is not installed, in that there are many restrictions in a usable range and supply because they need electricity, and in that calcium of the ceramic rod has a limit in dissolution of metal hydroxide generated by reacting with water. In the meantime, the hydrogen-rich water generator which generates hydrogen-rich water by reacting magnesium particles with drinking water has several problems in that a handle (hydrogen stick) containing the magnesium particles must be periodically inserted into drinking water contained in the plastic bottle specially designed, in that it is difficult in sanitary administration of the plastic bottle and the handle, and in that there is a limit in place and time to drink hydrogen-rich water.

Studies and developments on methods of minimizing generation of unnecessary gases by colon bacteria flora and maximizing generation of effective hydrogen gas by selectively taking oligosaccharides and polysaccharides existing in edible plants and materials extracted from edible spices are not yet known.

Additionally, methods of directly generating hydrogen gas inside the human body using characteristics of oligosaccharides and polysaccharides are not yet known.

Moreover, methods of storing hydrogen gas in a solidified hard candy or a soft film and taking the hard candy or the soft film are not introduced, and methods of storing gases, which have the same pressure and fluidity as hydrogen, in an airtight condition and supplying the gases into the human body in safety are also not yet known.

### SUMMARY OF THE INVENTION

The present invention provides a method of packing gases, a gas-containing vessel, and a technology of delivering gases into the human body or generating gases inside the human body.

Furthermore, the present invention provides a capsule for storing gases which is gradually dissolved and destroyed in water so that the gases contained in the capsule gradually leak out of the capsule.

Additionally, the present invention provides a capsule for storing hydrogen gas, a method of manufacturing the capsule and a method of packing hydrogen in the capsule in order to destroy airtightness of the capsule in prescribed time after a user takes the capsule containing hydrogen gas so that hydrogen gas leaks out of the capsule.

In addition, the present invention provides a method of manufacturing a hard hydrogen candy of a capsule type as a vessel for storing hydrogen.

Moreover, the present invention provides a method of manufacturing a gas-containing vessel, which can enhance a storage capacity per vessel by making a capsule of a low-caloric and soft film material to thereby reduce a caloric intake in contrast to hydrogen gas the user takes, and a method of charging and sealing the gas-containing vessel.

Furthermore, the present invention provides hydrogen-generating material, which can directly generate hydrogen gas inside the human body using characteristics of oligosaccharides and polysaccharides, and means and a method of directly packing hydrogen using the hydrogen-generating material and delivering the packed hydrogen to the inside of the intestine.

Additionally, the present invention provides a sugar material produced by selecting oligosaccharides and polysaccharides existing in edible plants and materials extracted from edible spices and properly adding and mixing them to a sugar material, in order to generate effective hydrogen gas by being fermented inside the large intestine by bacteria when the user takes.

In addition, the present invention provides a capsule type hydrogen storing vessel for generating lots of hydrogen through the steps of selectively and properly adding oligosaccharides and polysaccharides and materials extracted from edible spices, which generate hydrogen gas by being fermented by colon bacteria flora when the user takes, to the material of the candy vessel, and adding the hydrogen gas stored in the candy vessel to the hydrogen gas generated in the sugar vessel material to thereby generate lots of hydrogen, and a method of manufacturing the same

Moreover, the present invention provides a packing material, which is made into a polyethylene water jacket containing water or fire-resistant antifreeze and is melted and discharges water or fire-resistant antifreeze when temperature is above 120 degrees in order to pack, deliver and store the hydrogen-storing vessel or the hydrogen-generating candy vessel in safety.

The present invention provides a small-sized vessel being made of a material, which does not pass gases and is soluble in water, and having a sealed empty space in order to store gases therein. When water comes in contact with water, the airtightness of the vessel is gradually destroyed, and then, the gases contained in the vessel leak out of the vessel.

The gas-storing vessel according to the present invention can endure a change of atmospheric pressure in consideration of pressure applied to the inside and the outside of the vessel, and is 5 mm to 10 mm in external diameter, preferably 8 mm, is 8 mm to 20 mm in length of outer cylinder, preferably 14 mm, and is 10 mm to 25 mm in total length of the vessel including end plates 10 disposed at both ends, preferably 22 mm.

The gas-storing vessel is made with at least one compounds selected from saccharide, such as sugar, maltose, grain syrup and others, which are ingredients of soft sweets, amorphous hard sweets, and sugarfree hard sweets, carbohydrate syrup compounds of starch, maltitol, mannitol, sorbitol, Isomalt, xylitol and others, and compounds of polyols, and if necessary, at least on additional agent selected from gelatin, glycerin, ferrous fumarate, vitamin C, vitamin E, polyphenol, carotene, calcium, and others is added.

Gas stored in the vessel is hydrogen gas, and purity of hydrogen gas charged in the vessel is more than 99.999%, preferably, above 99.99999%.

As a method of sealing and packing hydrogen inside the smallest vessel, the method includes the steps of: molding a temporary vessel made of a material melted by heat and surrounding a hydrogen charging nozzle having a volume as big as hydrogen can be charged therein; mounting a thermocompression band at a portion excepting the length corresponding to the hydrogen charging space, removing air contained in the temporary vessel using the hydrogen charging nozzle, and charging hydrogen below the atmospheric pressure; and retreating the hydrogen charging nozzle to the end of the thermocompression band and sealing an entrance of the temporary vessel by heating the thermocompression band and tightening the band.

In the temporary vessel molding step, the vessel is made with massecuites, which are at least one compounds selected from saccharide, such as sugar, maltose, grain syrup and others, which are ingredients of soft sweets, amorphous hard sweets, and sugarfree hard sweets, carbohydrate syrup compounds of starch, maltitol, mannitol, sorbitol, Isomalt, xylitol and others, and compounds of polyols. The massecuites are made by adding at least one compounds selected from gelatin, glycerin, ferrous fumarate, vitamin C, vitamin E, polyphenol, carotene, calcium, and others, and is molded in a casting mold.

In the temporary vessel molding step, the vessel is made of a material, which has good airtightness, is low in calorific value, is thermally deformable when it is compressed by heat, and has glass transition temperature of more than 38° C. with water contents of more than 3%, and purity of hydrogen gas charged in the vessel is more than 99.999%.

The hydrogen gas charging system includes: a hydrogen charging nozzle part having hydrogen charging nozzles of n-number, which are inserted into the temporary vessel molded in a casting mold, and auxiliary devices; a net-type hydrogen charging pipe connected to the hydrogen charging nozzle part so as to become a path for vacuum gas or a path for hydrogen gas; a vacuum pumping part connected to the net-type hydrogen charging pipe for pumping the temporary vessel to make the temporary vessel in a vacuum condition; a hydrogen gas supply part connected to the net-type hydrogen gas charging pipe for supplying hydrogen gas and controlling a flow rate and pressure; a nitrogen gas supply part connected to the net-type hydrogen charging pipe for purging and stabilizing the hydrogen gas charging system.

A hard capsule which is arranged outside the gas-storing vessel and has an inner space larger than the volume of the gas-storing vessel is additionally provided to cover and seal the gas-storing vessel therein. Hydrogen gas stored in the gas-storing vessel below the atmospheric pressure first keeps sealability of hydrogen gas by the external atmospheric pressure and is secondarily provided with sealability of hydrogen gas by the hard capsule, so that the present invention can buffer a pressure change by a space formed between the gas-storing vessel and the hard capsule.

The present invention provides a hydrogen capsule which can be deeply inserted into the digestive organ of the human body because it store hydrogen gas in the smallest vessel and is orally eatable.

The soft film gas-storing vessel out of the gas-storing vessels is made with a soft film, which is dissolved in water but is not dissolved in organic solvent, and seals gas therein.

The gas is hydrogen, the vessel is molded in consideration of pressure change in atmospheric pressure, thickness of a cooled and dried film is 0.7 mm to 1.0 mm, thickness of the joint part is 0.6 mm to 0.95 mm, and thickness of the restored nozzle insertion hole is 0.7 mm to 1.0 mm.

The soft film is made of at least one compounds selected from general gelatin, succinic acid gelatin, glycerin, starch, sorbitol monostearate or sorbit group used as plasticizer or softener, polyglycytol syrups, sucrose, mannitol, xylitol, maltose, reduced maltose syrup, maltitol, polyethylene glycol, and The base of the soft film is at least one selected from refined water, black oxide of iron, red oxide, and so on.

Hydrogen gas in the vessel is 70% to 97% in volume, and at least one ingredient selected from soybean oil, safflower oil, purified fish oil, purified processed oil, sesame oil, red-pepper seed oil, wheat germ oil, grape seed oil, olive oil, rape seed oil, evening primrose oil, and fruit flavor oil is 3% to 30% in volume.

As method of charging and sealing hydrogen gas in a soft film gas-storing vessel, the method of charging and sealing hydrogen gas in a soft film gas-storing vessel includes the steps of: making a soft film temporary vessel by putting and sealing oil into a soft film, inserting a hydrogen charging nozzle into the temporary vessel, removing oil contained in the temporary vessel, and injecting hydrogen gas; and removing the hydrogen charging nozzle from the vessel and sealing the nozzle insertion hole, which is perforated in the temporary vessel by the hydrogen charging nozzle, with a grouting material. Here, the hydrogen charging nozzle has a sharp end like a needle, and oil of 3% to 30% is remained in the temporary vessel when the oil is removed from the vessel.

Based on the fact that hydrogen gas is generated in the large intestine of the human body, the material generating hydrogen gas in the large intestine is found and mixed with sweets. People can eat the mixed sweets or the mixed sweets can be made in the form of a vessel storing hydrogen gas. When a person eats the vessel, hydrogen is generated in the intestine of the person.

The present invention provides a hydrogen generating sweet produced by mixing a material, which generates hydrogen gas in the large intestine by colon bacteria flora, with sweets at the rate of 30% to 70% to the sweets.

The hydrogen generating sweet is made of at least one selected from lactose, lactulose, raffinose, stachyose, verbascose, curcumin, capsaicin, inulin, gingerrol, allicin, gluten, glutenin, and so on.

The present invention provides a method of producing hydrogen generating sweet including the steps of: making massecuites from which moisture of sweet is removed by boiling at least one compounds selected from polyols, such as maltitol, mannitol, sorbitol, Isomalt, and xylitol, at 150° C. to 200° C. under atmospheric pressure; selecting at least one from lactose, lactulose, raffinose, stachyose, verbascose, curcumin, capsaicin, inulin, gingerrol, allicin, gluten, glutenin, and so on and adding the selected one to the massecuites; and molding sweets by molding or jetting the massecuites.

Alternatively, the present invention provides a method of producing hydrogen generating sweet including the steps of: making massecuites from which moisture of sweet is removed by boiling at least one compounds; selecting at least one selected from lactose, lactulose, raffinose, stachyose, verbascose, curcumin, capsaicin, inulin, gingerrol, allicin, gluten, glutenin, and so on, and adding the selected one to the massecuites; molding a sweet vessel by molding or jetting the massecuites; and charging and sealing hydrogen gas in the sweet vessel.

The gas-storing vessel according to claim 1, wherein one selected from hydrogen gas, carbon monoxide, carbon dioxide, Xenon, and helium, which are medical gases, is stored in the cylindrical capsule type vessel.

In order to pack and carry and store the hydrogen-storing vessels and hydrogen generating sweets in safety, the hydrogen generating sweet vessel is made using polyethylene water jackets containing water or fire-resistant antifreeze and the packing material is melted above 120 degrees to thereby discharge water or fire-resistant antifreeze.

According to the present invention, gases can be charged, stored and delivered in a subminiature vessel which has a sealed empty space and whose airtightness is destroyed when it comes in contact with water.

When the user orally takes the hydrogen-storing vessel together with drinking water or drinks drinking water after dissolving the hydrogen-storing vessel in the drinking water, hydrogen gas is supplied to the inside of the human body.

The present invention can be free from limitation in time and place to take active hydrogen.

The method and apparatus for manufacturing the hydrogen-storing vessel can pack not only hydrogen but also other gases in a subminiature size.

When the hydrogen-storing vessel is manufactured by the apparatus and method according to the present invention, the user can easily control the quantity of intake of hydrogen gas, properly adjust supply of active hydrogen in contrast to active oxygen generated, and can be distributed at a lower price than prior arts.

When the soft film gas-storing vessel according to the present invention is used to the hydrogen-storing vessel, it can remarkably improve a storage capacity of hydrogen gas per vessel, enhance flexibility coping with the outside temperature, and reduce caloric intake.

The present invention can increase the supply of harmless hydrogen into the human body without using the conventional method of generating metal hydroxide when the user takes plastic-formed metals or water-reactive chemicals in order to generate hydrogen inside the human body.

The present invention enables the user to generate lots of useful hydrogen gases by controlling an amount of gases generated inside the intestine when the user usually takes foods.

Intake of the present invention is superior in body safety to intake of calcium hydride made through plastic-forming of calcium carbonate.

The present invention can solve problems of restriction in time and place and decrease of concentration that are not solved by electrolyzed reduced water, pouched hydrogen water, hydrogen-rich water of a stick form, and so on.

The present invention can increase a hydrogen intake efficiency by having a hydrogen supply capability larger than subminiature vessels using sugar or subminiature vessels using soft film, which have only a hydrogen-storing function.

The present invention can accurately measure quality and quantity because it is injected into the human body by the unit of capsule.

The packing material of the vessel of the hydrogen-generating candy can prevent explosion even though hydrogen contained in the vessel leaks out due to exposure of the hydrogen-storing vessels to fire or due to carelessness.

### BRIEF DESCRIPTION OF THE DRAWINGS

FIGS. 1(a) and 1(b) are sectional views of a hydrogen-storing vessel accommodating a gas-storing vessel/hard capsule according to a preferred embodiment of the present invention.

FIGS. 2(a) to 2(e) are flow diagrams showing processes of molding the gas-storing vessel and charging hydrogen gas according to the present invention.

FIGS. 3(a) and 3(b) are schematic diagrams of a hydrogen gas charging system according to the present invention.

FIG. 4 shows a sectional view and a front view of the soft capsule (soft film gas-storing vessel) according to the present invention.

FIGS. 5 and 6 are process charts for explaining a method of injecting and sealing hydrogen according to the present invention.

FIG. 7 is a structural diagram of a grouting nozzle and a solid bonding bar to seal the storing vessel after an gas injection.

FIG. 8 shows a plan view and a side view of a water jacket of a vessel for storing a hydrogen generating candy according to the present invention.

### EXPLANATION OF ESSENTIAL REFERENCE NUMERALS IN DRAWINGS

1: hydrogen-storing vessel 2: gas-storing vessel
16: hard capsule 6: cylindrical vessel 7: hydrogen gas
21: temporary vessel 22: upper-sealed vessel
25: hydrogen charging nozzle
26: round thermocompression band 41: oil pump
42: pumping oil control valve
44: oil discharge valve (drain valve)
45: pumping oil separation valve
46: net-type pumping oil pipe
62: hydrogen gas 51: soft film gas-storing vessel
52: outer part of vessel
53: inner part of vessel
54: joint part of vessel 55: outer length of vessel
56: outer diameter of vessel
57: restored nozzle insertion hole
58: oil remainder 61: soft film temporary vessel
62: oil content 63: hydrogen charging nozzle insertion hole
64: hydrogen charging nozzle
65: damaged nozzle insertion hole
66: grouting nozzle 71: melted bonding rod injection part
72: bonding rod heating part (heater)
73: bonding rod supporting part
74: bonding rod of solid phase
81: unit water jacket 82: convex part of water jacket
83: concave part of water jacket 84: female hook
85: male hook

### DETAILED DESCRIPTION OF THE INVENTION

Reference will be now made in detail to the preferred embodiments of the present invention with reference to the attached drawings.

Hydrogen charged in a hydrogen-storing vessel enabling hydrogen to be absorbed into the human body has purity of more than 99.000% which is absorbable and metabolizable inside the human body. It is good that a rate of impurities in hydrogen is lower than concentration of the following Table 1.

TABLE 1

| Item | O₂ | H₂O | CO | CO₂ | CH₄ | N₂ | Ar |
|---|---|---|---|---|---|---|---|
| Impurities in hydrogen | 0.2 | 2 | 0.2 | 0.2 | 0.2 | N/A | N/A |
| gas (ppm) | | | | | | | |
| Impurities in refined | 10 | 10 | 10 | 10 | 10 | 10 | 10 |
| hydrogen (ppb) | | | | | | | |

In the above table, it is good that one or less dust of less than 0.1 micrometer (um) per 1 cu ft(ft³) is contained in the hydrogen gas.

Hydrogen gas is a combustible material, has the ignition point at 550° C., and must be stored and used within a safe range because it is a colorless, odorless, tasteless, nontoxic and suffocating gas which explodes within a limitation of a volume ratio ranging 4% to 74% in the air and within a limitation of a volume ratio ranging 18% to 59% in a limited space. Hydrogen atoms can be easily separated from water molecules because they have the smallest and lightest diameter out of all atoms, rapidly spread in the air and disappear into the air, and can pass through cells of the skin of the human body.

According to a theory found through studies on hydrogenase, recently, it has been revealed that hydrogenase of bacteria existing in microorganisms in the soil is an enzyme promoting reversible oxidation of hydrogen molecules, plays an important role in anaerobic metabolism, makes hydrogen molecules, decomposes hydrogen molecules into hydrogen ions and electrons, and has a mechanism of using iron as catalyst.

Hydrogen molecules (H₂) is oxidized and hydrogen-ionized while giving electrons to electron acceptors, such as oxygen (O₂), carbon dioxide (CO₂), and so on. It is known that low molecular weight compounds and protein, such as ferredoxins and Cytochrome c, may be physically electron donors and may be electron acceptors in relation with hydrogenase.

H₂+Aₒₓ→2H⁺+A_{red}

2H⁺+D_{red}→H₂+Dₒₓ

It is known that Enzymes related with electron delivery have to essentially keep low oxidation-reduction potential (ORP) for anaerobic metabolism.

Hydrogen gas contained in the hydrogen-storing vessel, which is put into the mouth like drinking water, or, which a user takes after dissolving in drinking water, is hydrogen molecules and is decomposed by hydrogenase existing in bacteria of microorganisms existing in the human body during metabolism to thereby be separated into hydrogen ions and electrons. The separated hydrogen ions are active hydrogen atoms functioning to remove active oxygen.

In general, it is known that about 2% of oxygen inhaled into the lungs is converted into active oxygen. A volume of oxygen inhaled for one minute is about 2,000 cc, and a volume of oxygen converted into active oxygen out of the inhaled oxygen is 40 cc for one minute and 57,600 cc for 24 hours. In the meantime, According to Hayashi Hidemitsu's book, “Revolution of water, hydrogen-rich water”, dissolved hydrogen of hydrogen-saturated water that can be dissolved in tap water to the maximum is 1.49 ppm, and dissolved hydrogen of hydrogen-rich water by a magnesium stick is 1,203 ppm, and hence, any type of hydrogen-rich water has a volume limit in supply of hydrogen gas into the human body. The hydrogen-storing vessel according to the present invention provides a method of supplying hydrogen gas by setting a ration of active hydrogen to active oxygen higher than hydrogen-rich water.

In order to inhale hydrogen to the human body, it is necessary to develop a proper hydrogen gas packing vessel for oral administration. Moreover, when a predetermined period of time passes after the oral administration, hydrogen gas reaches the intestine, and in this instance, a vessel for making hydrogen leak out of the vessel and spread to the human body, namely, a vessel that keeps airtightness for a predetermined period of time and gradually breaks the airtight condition, is needed. In order to satisfy the needs, a capsule type sweet vessel is selected.

Sweet which is a material for the capsule type vessel for storing hydrogen gas must store hydrogen gas therein in safety for a long time in changes of temperature and pressure.

Ingredients of the sweets are compounds, which are selected from conventional soft sweets, amorphous hard sweets, sugarfree hard sweets, and others and are produced according to the conventional sweet producing method, and the compounds provide a perfect sealability of air or nitrogen gas, provide a good sealability of hydrogen gas, have a calorific value of less than 10 Kcal per one capsule, are high in tensile strength, are thermally deformable during the process of manufacturing the hydrogen-storing vessel, have glass transition temperature of more than 38° C. with water contents of more than 3%, and are similar to glass tissue in composition at room temperature.

Ingredients of the conventional sweets which are applicable to the compounds are at least one compound selected from saccharide, such as sugar, maltose, grain syrup and others, carbohydrate syrup compounds of starch, maltitol, mannitol, sorbitol, Isomalt, xylitol and others, and compounds of polyols. Moreover, gelatin, glycerin, and others which are used as ingredients of a capsule may be used as addition agent.

When massecuites which are made by the above ingredients and from which moisture is removed more or less are put and molded in a capsule-shaped casting mold whose upper portion is opened, a cylindrical thin space which is opened at one side is formed inside the sweet. The space is in a vacuum condition, and then is filled with low pressure hydrogen gas and compressed and sealed, so that the hydrogen-storing vessel in which hydrogen gas is stored is completed. If the structure and tissue of sweet are similar those of glass, outside air cannot penetrate to the inside but some of hydrogen molecules may leak out of the sweet but do not leak at internal pressure lower than atmospheric pressure. In order to store hydrogen gas in safety, the gas-storing vessel is accommodated in a general hard capsule with excellent sealability to thereby secondarily enhance sealability, and then, put and thermally compressed in a pocket-type packing structure having upper and lower aluminum layers to thereby thirdly enhance sealability and completely manufacture the hydrogen-storing vessel.

The hard sweets are generally called hard sweets or hard boiled candies. They are amorphous and hard confectioneries and are obtained by strongly dehydrating carbohydrate syrups. The amorphous hard sweets which are sugarfree or contain polyols may be also obtained using carbohydrate syrups containing hydrogen or carbohydrate syrups digested a little. The sugarfree hard sweets gradually come into the spotlight, and the reason is that the sugarfree hard sweets have similar organoleptic property but restrict tooth decay and are lower in calories than general sugar hard sweets. Generally, the sugarfree hard sweets are produced by boiling compounds of polyols melted in water. The compounds of polyols are boiled to the range of 150° C. to 200° C. under atmospheric pressure such that most of moisture is evaporated. In order to reduce water content more, the boiling process is carried out in a vacuum condition, and in this instance, normal water content is 2.5% or less and can be lowered to 1.5% or less. After that, various materials, for instance, spices, pigments, strong sweeteners, acids, plant extracts, vitamins, and active medicines, are added to the massecuites obtained through the above. After that, the massecuites to which various materials are added are cooled and put into the casting mold to form the shape. Alternatively, the shape of the sugarfree hard sweets may be formed on a roll or by extrusion. The technology of the hard sweets is described in Korean Patent No. 0163252.

Hereinafter, referring to the drawings, the hydrogen-storing vessel and the method of manufacturing the hydrogen-storing vessel according to the present invention will be described.

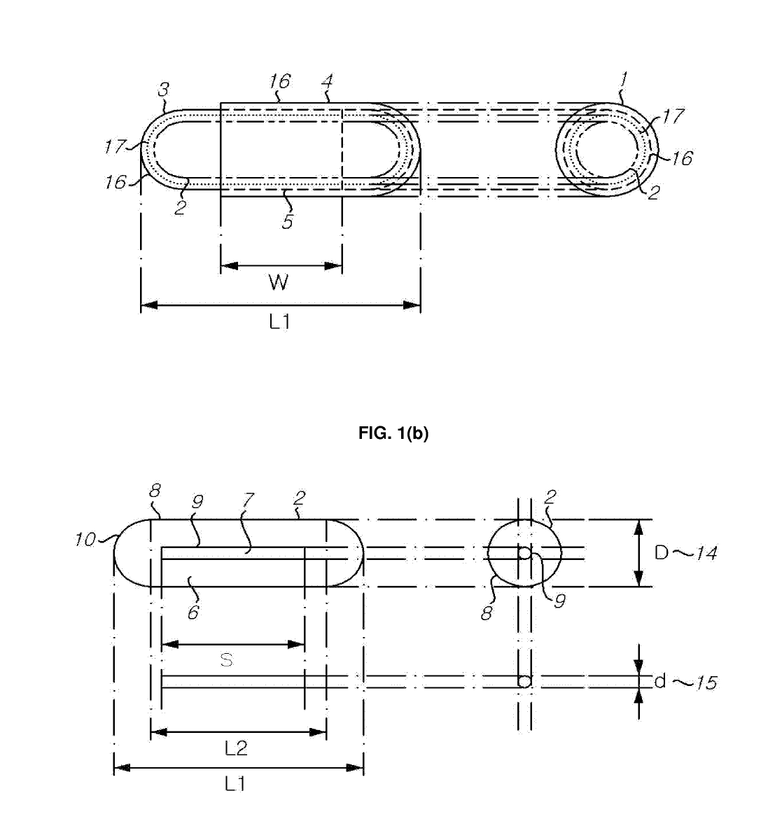

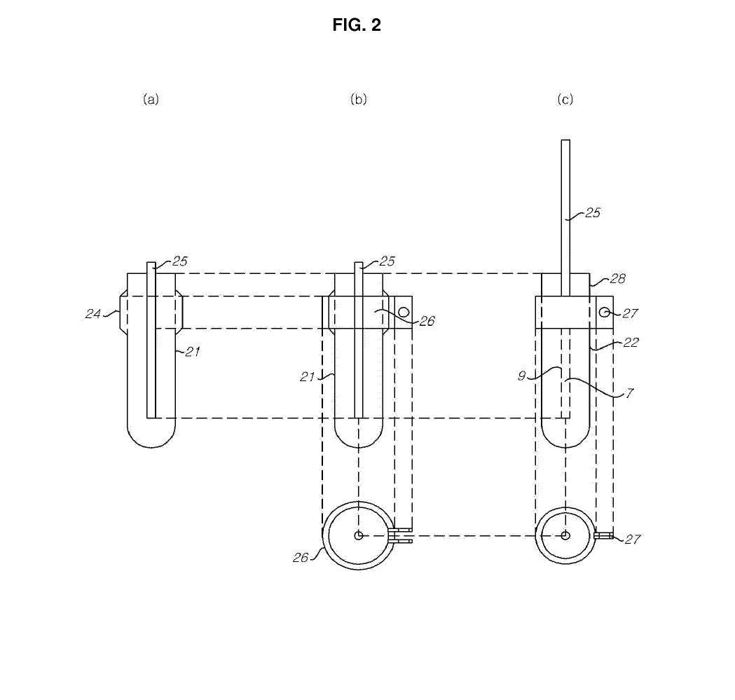

In FIGS. 1 and 2, the same parts have the same reference numerals, and their detailed description will be omitted.

### Embodiment 1

As shown in FIG. 1a, the gas-storing vessel according to the present invention is a sweet capsule for storing hydrogen gas. The gas-storing vessel 2 includes a hard capsule 16 having a space therein. The gas-storing vessel 2 is put to the inside of the hard capsule 16, and then, an upper part 4 of the hard capsule and a lower part 3 of the hard capsule are bonded together under a nitrogen gas atmosphere. Accordingly, the hard capsule has a hydrogen-storing vessel 1 having a bonded portion 5.

As shown in FIG. 1 b, the gas-storing vessel 2 includes a cylindrical vessel body 6, an outer cylindrical part 8 of the vessel, an inner cylindrical part 9 of the sweet, and hemispherical end plates 10 formed integrally to both ends of the outer cylindrical part 8. A hydrogen gas 7 which has pressure lower than atmospheric pressure is stored in the inner cylindrical part 9, and then, the gas-storing vessel 2 is manufactured in an orally edible size.

In consideration of pressure applied to the inside and the outside of the cylindrical vessel body 6, with a tensile strength that can endure 1.1 times higher than the atmospheric pressure, an outer diameter (D,14) of the vessel body 6 does not exceed 8 mm, a length (L2) of the outer cylinder does not exceed 14 mm, and the total length (L1) of the entire vessel to which the end plates 10 disposed at both ends are added does not exceed 22 mm.

On the supposition that development of pressure change of the hydrogen gas, which is stored in the hydrogen-storing vessel 1, according to changes in external temperature is ideal gas, it is illustrated in the following Table 2. On the supposition of the ideal gas, the hydrogen gas charged under the condition, 1 atmospheric pressure and 15° C., is 0.87 kg/cm² at 30 degrees below zero and 1.0987 kg/cm² at 30 degrees above zero in order to cope with the temperature change. Accordingly, a vessel and capsule space 17 is formed between the gas-storing vessel 2 and the hard capsule 16, and has an interval at more than 1% of the outer diameter 14 of the cylindrical vessel body. When pressure of the inner cylindrical part 9 is higher than pressure of the outer cylindrical part 8, some of the hydrogen gas 7 stored in the inner cylindrical part 9 passes and goes through the outer cylindrical part 8, the hydrogen gas 7 is locked together with nitrogen in the vessel and capsule space 17. On the contrary to the above, when pressure of the inner cylindrical part 9 is lower than pressure of the outer cylindrical part 8, a reversible penetration that the locked hydrogen gas returns to the inner cylindrical part 9 occurs, so that the change in pressure applied to the gas-storing vessel 2 can be absorbed to thereby absorb shock.

TABLE 2

| | ° C. | | | | | | |
|---|---|---|---|---|---|---|---|
| | −45 | −30 | −15 | 0 | 15 | 30 | 45 |
| K | 228.15 | 243.15 | 258.15 | 273.15 | 288.15 | 303.15 | 318.15 |
| Charging pressure of | | | | 0.98 | | | |
| hydrogen gas (kg/cm²) | | | | | | | |
| Pressure change of | 0.82 | 0.87 | 0.93 | 0.98 | 1.03 | 1.09 | 1.14 |
| hydrogen gas (kg/cm²) | | | | | | | |
| Volume change of inner | | | | 0.95 | 1.00 | 1.05 | 1.10 |
| cylindrical part(15° C.~1) | | | | | | | |
| Diameter change of outer | | | | | 1.00 | 1.03 | 1.05 |
| cylindrical part(15° C.~1) | | | | | | | |
| Necessary interval between | | | | | 0 | 2 | 3 |
| capsule and sweet, % of | | | | | | | |
| diameter of outer cylindrical part | | | | | | | |

Ingredients of the sweets are compounds, which are selected from conventional soft sweets, amorphous hard sweets, sugarfree hard sweets, and others and are produced according to the conventional sweet producing method, and the compounds provide a perfect sealability of air or nitrogen gas, provide a good sealability of hydrogen gas, have a calorific value of less than 10 Kcal per one capsule, are high in tensile strength, are thermally deformable during the process of manufacturing the hydrogen-storing vessel, have glass transition temperature of more than 38° C. with water contents of more than 3%, and are similar to glass tissue in composition at room temperature.

Ingredients of the conventional sweets which are applicable to the compounds are at least one compound selected from saccharide, such as sugar, maltose, grain syrup and others, carbohydrate syrup compounds of starch, maltitol, mannitol, sorbitol, Isomalt, xylitol and others, and compounds of polyols. Moreover, gelatin, glycerin, and others which are used as ingredients of a capsule may be used as addition agent.

Hydrogen charged in a hydrogen-storing vessel 2 has purity of more than 99.999%, and one or less dust of less than 0.1 micrometer per 1 ft³ is contained in the hydrogen gas. In the case that the hydrogen gas 7 is charged at pressure lower than the atmospheric pressure, air or nitrogen gas introduced from the outside of the gas-storing vessel 2 is cut off, but the hydrogen gas stored in the gas-storing vessel 2 may leak out of the hydrogen-storing vessel 6 a little when pressure is higher than atmospheric pressure, but does not leak when pressure of the hydrogen gas stored in the gas-storing vessel is equal to or lower than atmospheric pressure.

### Embodiment 2

FIG. 2 is a flow diagram showing a method of charging hydrogen gas into the gas-storing vessel 2.

a) Massecuites which are made with saccharide, compounds of carbohydrate syrups, and compounds of polyols according to selection of materials of the conventional sweet and from which moisture is removed more or less are prepared. After that, additional agents respectively necessary for the massecuites are put to the massecuites, and then the massecuites are cooled and put into a casting mold as indicated in the drawing to make a shape. The additional agents may include ferrous fumarate for decomposing the hydrogen molecules into hydrogen atoms (active hydrogen), vitamin C serving to help antioxidation, vitamin E, polyphenol, carotene, calcium, and so on. It is preferable that ferrous does not exceed 4 mg per capsule.

The casting mold of the vessel is the same as the conventional sweet molding method. The casting mold has a shape and a structure that a temporary vessel 21 of FIG. 2(a) can be molded. Moreover, the temporary vessel 21 has a lower part of a capsule shape, an upper part having an upper protrusion 24 which is compressible by a round thermocompression band 26, and a central part molded in a state where a lower portion of a hydrogen charging nozzle 25 made of metal is inserted. A length of the temporary vessel 21 is equal to the sum of the total length (L1) of the vessel and a height of an upper finishing portion 28 of the vessel, and a width of the round thermocompression band 26 is equal to a thickness of the end plate 10 of the vessel.

b) The upper protrusion 24 of the temporary vessel 21 is filled with the round thermocompression band 26, the inside of the hydrogen charging nozzle 25 is pumped to the degree of vacuum of about 10⁻¹ Torr by a vacuum pump, and then, hydrogen gas is charged from a hydrogen gas supply part at pressure lower than atmospheric pressure.

c) The hydrogen charging nozzle 25 raises temperature above the glass transition temperature by a built-in heater, and retreats to an end portion of the vessel as long as the sum of a length (l) of the inner cylindrical part and a width of the round thermocompression band 26. In this instance, a cylindrical length of the temporary vessel 21 is equal to the sum of the length (l) of the inner cylindrical part and the width of the round thermocompression band 26. When the hydrogen charging nozzle retreats, temperature is raised above the glass transition temperature that can transform the upper protrusion 24 of the vessel by the heater mounted on the round thermocompression band 26, so that the thermocompression band 26 is completely compressed to the length corresponding to the circumference of the lower part of the temporary vessel 21 and the width of the round thermocompression band 26 and is tightened till be sealed by a tightening part 27 of the thermocompression band 26. When tightening for sealing is finished, the inner cylindrical part 9 of the vessel is made inside the temporary vessel 21, and only the upper finishing portion 28 of the vessel remains. In order to prevent cracks of the vessel during the process of compressing and sealing the upper protrusion 24, temperature and moisture of the temporary vessel 21 is controlled by a controller.

d) As shown in FIG. 2(d), the inner cylindrical part 9 of the vessel 2 whose upper portion is sealed is charged with the hydrogen gas 7 as much as the length (l) of the inner cylindrical part at pressure below the atmospheric pressure. When the upper portion of the outer cylindrical part 8 is as high as the sum of the thickness of the end plate 10 and the upper finishing portion 28, the hydrogen charging nozzle 25 is completely pulled out, the round thermocompression band 26 is removed, and gaps which are not inevitably sealed are grouted with the ingredients of the sweet.

e) While heat is applied till the upper finishing part 28 of the sealed vessel 22 reaches temperature to transform the upper finishing part 28, the upper finishing part 28 is removed remaining as much as the thickness of the end plate 10, and then, as shown in FIG. 2(e), the end plate 10 of the upper part is finished in a hemispherical shape, whereby the gas-storing vessel 2 illustrated in FIG. 1 is perfectly manufactured.

### Embodiment 3

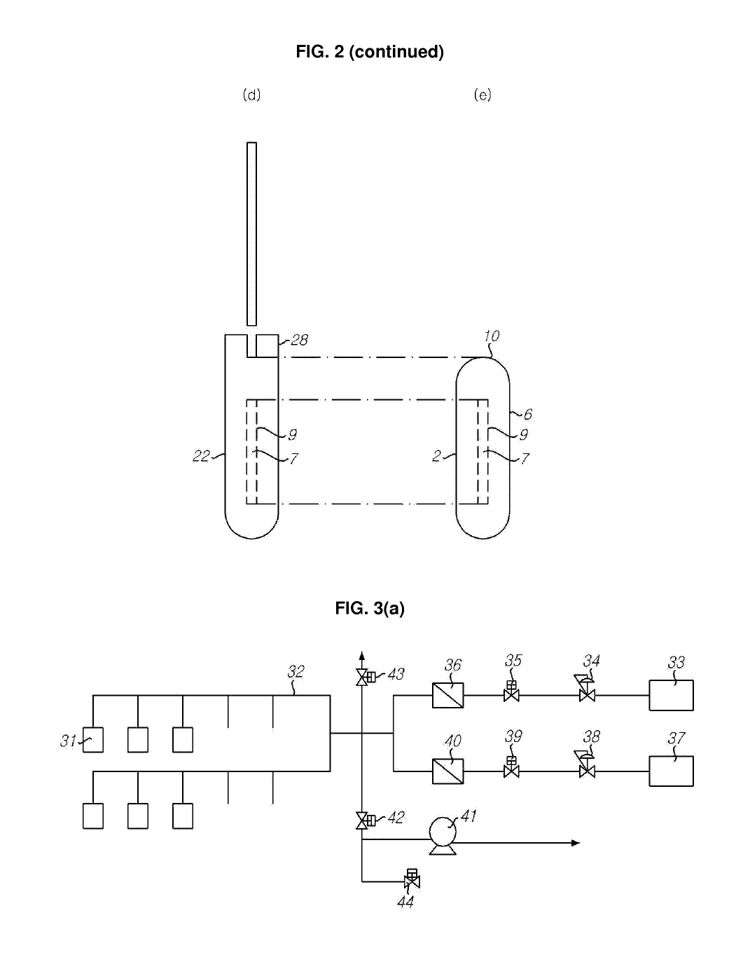

FIG. 3 is a schematic diagram of a hydrogen gas charging system according to the present invention. As shown in FIG. 3a, based on that a net-type hydrogen charging pipe connected with a hydrogen charging nozzle part is used for pumping and charging, the hydrogen gas charging system will be described.

The hydrogen gas charging system includes: a hydrogen charging nozzle part 31 having hydrogen charging nozzles 25 of n-number and auxiliary devices; a net-type hydrogen charging pipe 32 connected to the hydrogen charging nozzle part 31; a vacuum pumping part having a vacuum pump 41 and a pumping gas control valve 42 connected to the net-type hydrogen charging pipe 32; a hydrogen gas supply part connected to the net-type hydrogen gas charging pipe 32 and having a hydrogen gas supply source 33, a hydrogen gas pressure regulator 34, a hydrogen gas control valve 35, and a hydrogen gas filter 36; a nitrogen gas supply part connected in parallel to the net-type hydrogen charging pipe 32 together with the hydrogen gas supply part and having a nitrogen gas supply source 37, a nitrogen gas pressure controller 38, a nitrogen gas control valve 39, and a nitrogen gas filter 40; and a pumping gas exhaust valve 44 for exhausting pumping gas and a supply gas exhaust valve 43 for purging supply gas.

FIG. 3(b) illustrates the case that the net type hydrogen charging pipe connected with the hydrogen charging nozzle part and the net-type pumping gas pipe are used separately. In FIG. 3(b), the hydrogen gas charging system includes: a hydrogen charging nozzle part 31 having hydrogen charging nozzles 25 of n-number and auxiliary devices; a vacuum pumping part having a vacuum pump 41 and a pumping gas control valve 42 connected to the net-type pumping gas pipe 46 connected with the hydrogen charging nozzle part 31, in the case that the hydrogen charging nozzle part 31 and the net type hydrogen charging pipe 32 are separated from each other by pumping gas separation valves 45 of n-number; a hydrogen gas supply part connected to the net-type hydrogen gas charging pipe 32, in the case that the hydrogen charging nozzle part 31 and the net type hydrogen charging pipe 32 are connected to each other by the pumping gas separation valves 45 of n-number; a nitrogen gas supply part connected in parallel to the net-type hydrogen charging pipe 32 together with the hydrogen gas supply part; a pumping gas exhaust valve 44 for exhausting pumping gas; and a supply gas exhaust valve 43 for purging supply gas.

In the hydrogen gas charging system according to the present invention, the charging process illustrated in FIG. 3(a) will be described. In a state where the hydrogen gas control valve 35, the nitrogen gas control valve 39, the supply gas exhaust valve 43, and the pumping gas control valve (39), the supply gas exhaust valve 43 and the pumping gas control valve 42 are closed and the pumping gas exhaust valve 44 is opened, the vacuum pump 41 is operated. After that, the pumping gas exhaust valve 44 is closed and the pumping gas control valve 42 is opened, so that the net-type hydrogen charging pipe 32 and the hydrogen charging nozzles 25 of the hydrogen charging nozzle part 31 are pumped to thereby increase the degree of vacuum to 10⁻¹ Torr. When the hydrogen charging nozzles 25 and their paths reach the degree of vacuum of a prescribed level, the pumping gas control valve 42 is closed and the hydrogen gas control valve 35 of the hydrogen gas supply part is opened, so that hydrogen gas is charged from the hydrogen gas supply source 33 through the hydrogen gas pressure regulator 34 and the filter 36 till the hydrogen charging nozzles 25 reach the atmospheric pressure. When the hydrogen gas charging is finished, the hydrogen gas control valve 35 is closed to stop the supply, and then, the upper protrusions 24 of the hard sweets are compressed according to the thermal compression process as described above by the round thermocompression band 26 to thereby seal the charged hydrogen gas.

Nitrogen gas of the nitrogen gas supply part is to use when a user wants to purge the inside of the charging system at the time of organization or emergency. When the nitrogen gas control valve 39 is opened, nitrogen gas is supplied from the nitrogen gas supply source 37 through the nitrogen gas pressure regulator 38 and the filter 40. Nitrogen gas may be used to seal nitrogen contained inside the system during a pause of facilities. The supply gas exhaust valve 43 opens a valve to exhaust hydrogen gas or exhaust the nitrogen gas if necessary.

In the hydrogen gas charging system according to the present invention, the charging process shown in FIG. 3(b) will be described. The pumping gas separation valve 45 is closed to be separated from the net-type hydrogen charging pipe 32. In the state where it is separated from the net-type hydrogen charging pipe 32, the pumping gas control valve 42 is closed and the pumping gas exhaust valve 44 is opened so as to operate the vacuum pump 41. After that, after the pumping gas exhaust valve 44 is closed and the pumping gas control valve 42 is opened, so that the net-type pumping gas pipe 46 and the hydrogen charging nozzles 25 of the hydrogen charging nozzle part 31 are pumped to increase the degree of vacuum to 10⁻¹ Torr. When the inside of the hydrogen charging nozzles 25 and their paths reach a predetermined degree of vacuum, the pumping gas control valve 42 is closed and the pumping gas separation valves 45 are closed to thereby communicate with the net-type hydrogen charging pipe 32. After that, the hydrogen gas control valve 35 of the hydrogen gas supply part is opened, so that hydrogen gas is charged from the hydrogen gas supply source 33 through the hydrogen gas pressure regulator 34 and the filter 36 till the hydrogen charging nozzles 25 reach the atmospheric pressure. When the hydrogen gas charging is finished, the hydrogen gas control valve 35 is closed to stop the supply of hydrogen gas, and then, the lower portion of the upper protrusion 24 of each sweet is compressed by the round thermocompression band 26 according to the thermocompression process to thereby seal the charged hydrogen gas. The function of the nitrogen gas supply part is the same as the above.

The essential points of the manufacturing process will be adjusted as follows.

Massecuites which are formed by removing moisture from raw materials of the sweet are prepared, additional agents are added to the massecuites, and then, the massecuites are cooled. A casting mold for sweets is prepared. After that, the hydrogen charging nozzles are mounted at the central portion to a set height, and the massecuites are put into the casting mold in a state where the hydrogen charging nozzles are inserted into the inside of the casting mold to thereby mold a temporary vessel.

The round thermocompression band is charged in the upper protrusion of the temporary vessel, the inside of the hydrogen charging nozzle is pumped in a vacuum condition to thereby be charged with hydrogen gas. After that, when temperature of the hydrogen gas charging nozzle is raised in such a fashion that the end portion of the upper protrusion moves back, the inside of the temporary vessel is formed.

When the upper protrusion is compressed and tightened to be sealed by the round thermocompression band, it makes a vessel whose upper portion is sealed.

When the hydrogen charging nozzle is completely moved out, the round thermocompression band is removed, and if necessary, grouted, and then, when the upper finished portion of the upper-sealed vessel is removed and the upper portion is finished to become an end plate, manufacturing of the gas-storing vessel is finished.

When the gas-storing vessel is put into a hard capsule and bonded and sealed under a nitrogen atmosphere, manufacturing of the hydrogen-storing vessel according to the present invention is finished. The completed hydrogen-storing vessel is packed and treated by a pocket-type medicine structure having aluminum layers at an upper portion and a lower portion.

### Embodiment 4

A method of manufacturing a not hard but soft gas-storing vessel through a more improvement of the gas-storing vessel will be described.

Materials used for manufacturing a temporary gas-storing vessel of a soft film are compounds of at least one selected from general gelatin extracted from natural collagel, succinic acid gelatin for increasing decomposition stability by a carboxylic group converted from an amino group, glycerin, concentrate glycerin, starch, sorbitol monostearate or sorbit group used as plasticizer or softener, polyglycytol syrups, sucrose, mannitol, xylitol, maltose, reduced maltose syrup, maltitol, polyethylene glycol, and so on. At least one selected from refined water, black oxide of iron, and red oxide of iron is mixed to the compounds, and then, a gel mass of the film is prepared through swelling, dissolution and deformation.

Because the contents of the temporary vessel of the soft film will be substituted with hydrogen gas after the vessel is manufactured, oil is used as a material that can give stability even though there is no surfactant between the film and the contents. An oil base is at least one selected from soybean oil, safflower oil, purified fish oil, purified processed oil, sesame oil, red-pepper seed oil, wheat germ oil, grape seed oil, olive oil, rape seed oil, evening primrose oil, fruit flavor oil, and so on.

The method of manufacturing the temporarily soft-film vessel, like the conventional manufacturing method, includes the steps of putting the contents at the center of two film bases, and bonding the two films in an oval shape, so that the soft temporary vessel which seals the contents is made.

A film of a uniform thickness is made in a general rotary type automatic charger (molding machine), is cooled by cold air with relative humidity of about 20% to 30% at about 12° C. to 20° C. to thereby be gelled, and then, is delivered to the molding machine. Contents are charged through the charger by a small amount.

Films located at both ends of the spread box of the charger are uniformly drawn, and then, both sides of the films are sewed and adhered by mechanical pressure and electric heat (35° C. to 40° C.) of a die roll, such that characteristics of the film are not transformed due to leakage of the contents or imbalance of moisture at the seam of the sewed film.

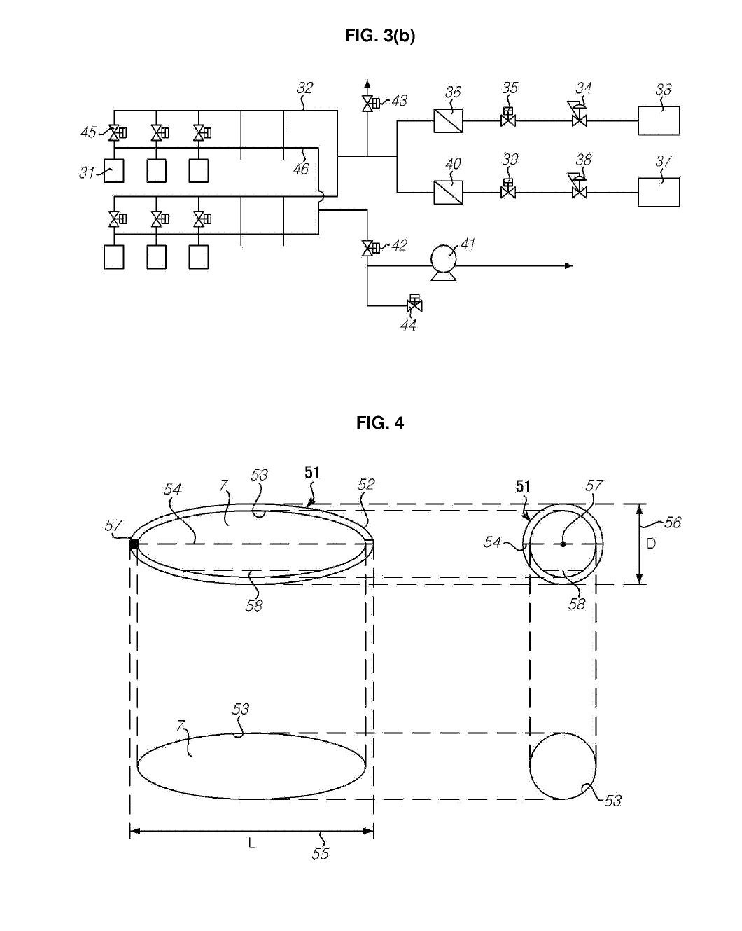

FIG. 4 is a sectional view of the gas-storing vessel of the soft film. As shown in FIG. 4, the gas-storing vessel has the outer appearance like a rugby ball which becomes an oval shape in the longitudinally sectional view and becomes a round shape in the horizontally sectional view.

The gas-storing vessel is a soft film gas-storing vessel 51 which has a soft film formed in a capsule type and in which hydrogen gas 7 of a predetermined pressure is contained as the base of the contents and a small amount of oil remainder 58 is stored. The gas-storing vessel 51 may be accommodated in a hard capsule having an inner space for accommodating the gas-storing vessel therein to protect the soft film gas-storing vessel.

The soft film gas-storing vessel 51 includes an outer part 52 of the vessel, the inner part 53 of the vessel, and a joint part 54 of the vessel, and a nozzle insertion hole 57 at which the end plate portion damaged while charging hydrogen is restored. The hydrogen gas 7 which does not exceed twice the atmospheric pressure and oil remainder 58 ranging 3% to 30% are stored in the inner part 53 of the vessel, and the external length (55:L) of the vessel and the external diameter (56:D) of the vessel are made to have sizes for allowing the user to orally put the gas-storing vessel into the mouth.

The dissolution time of the soft film gas-storing vessel is within 12 minutes when the soft film gas-storing vessel is reinforced by the hard capsule, or is within 30 minutes when the soft film gas-storing vessel is only took into the user's mouth.

In consideration of pressure applied to the inside and the outside of the gas-storing vessel, the thickness of the film of the temporary soft film vessel 61 is 0.8 mm to 1.1 mm at the time of molding, but is 0.7 mm to 1.0 mm after being cooled and dried through the general method, so that the gas-storing vessel has tension or elasticity to endure 1.1 times the pressure. The thickness of the joint portion 54 of the vessel is 0.6 mm to 0.95 mm after being cooled and dried, and the thickness of the restored nozzle insertion hole 57 is 0.7 mm to 1.0 mm after being cooled and dried.

The method of manufacturing the soft film gas-storing vessel includes a process of molding the soft film gas-storing vessel and a process of charging and sealing hydrogen gas.

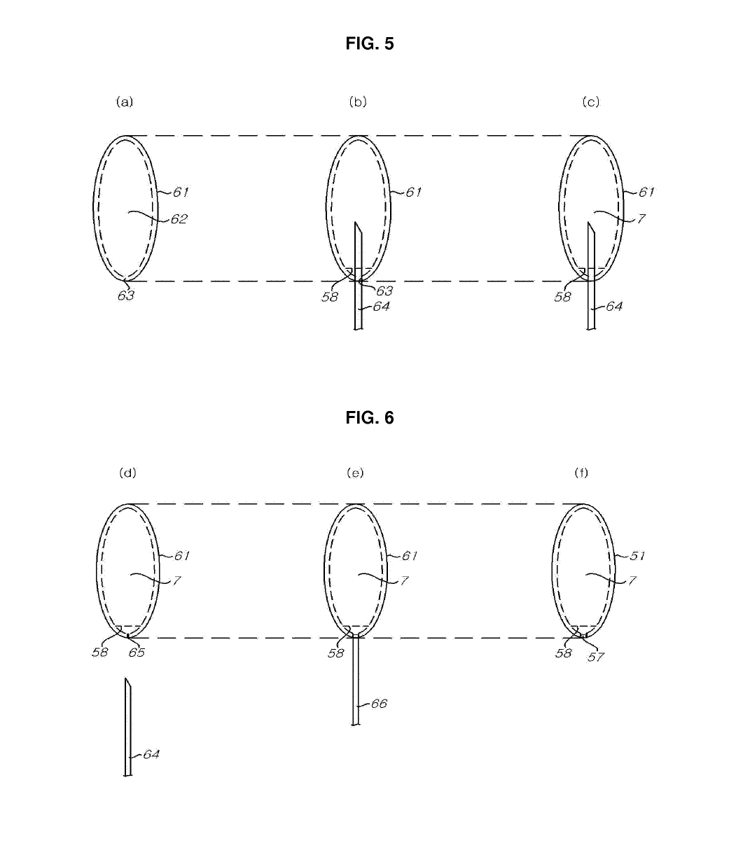

FIG. 5 is a process chart for explaining the process of molding the soft film gas-storing vessel 61, like the conventional technology, including the steps of manufacturing a soft capsule in which oil is sealed, discharging the oil of the soft capsule, and injecting hydrogen gas.

First, as shown in FIG. 5(a), the soft film gas-storing vessel 51 is manufactured when hydrogen gas is charged and sealed in the subminiature temporary soft film vessel 61. In this instance, a predetermined thickness is provided to the ingredients of the soft film generally used in manufacturing soft capsules so as to reinforce tension and elasticity, and the temporary soft film vessel 61 having the joint portion 54 of the vessel is made in such a fashion that the vessel is filled with oil contents 62 and sewed.

Next, as shown in FIG. 5(b), in a state where the temporary vessel 61 is located vertically, the hydrogen charging nozzle insertion hole 63 is formed at the joint portion 54 formed at the end plate part of the lower part of the vessel, and the hydrogen charging nozzle 64 of a sharp needle form is inserted into the central portion of an oil depth inside the temporary vessel 61 to the corresponding depth upwardly from the bottom through the nozzle insertion hole 63.

Continuously, as shown in FIG. 5(c), after sealability of the hydrogen charging nozzle insertion hole 63 is checked, the oil contents 62 stored inside the temporary vessel 61 are pumped and discharged through the hydrogen charging nozzle while the hydrogen charging nozzle 64 moves inside the vessel to the length corresponding to the depth of the oil remainder. After that, the oil remainder 58 existing in the temporary vessel, which remains after the hydrogen gas is charged, covers 3% to 30% of the bottom of the vessel, and then, the hydrogen gas 7 is charged till reaching a predetermined pressure. In this instance, the charged pressure does not exceed twice the atmospheric pressure.

Next, as shown in FIG. 5(d), while the hydrogen charging nozzle 64 completely moves out through the nozzle insertion hole 63 of the temporary vessel 61, the damaged nozzle insertion hole 65 can temporarily keep sealability by elasticity of a sealing membrane formed by the oil remainder 58 and elasticity of the film.

Moreover, as shown in FIG. 5(e), in a state where the temporary vessel 61 temporarily keeps sealability, while the materials of the soft film are injected into the damaged nozzle insertion hole 65 through the grouting nozzle 66, grouting is carried out at predetermined injection pressure and electric temperature (below 200° C.), so that the damaged nozzle insertion hole is bonded.

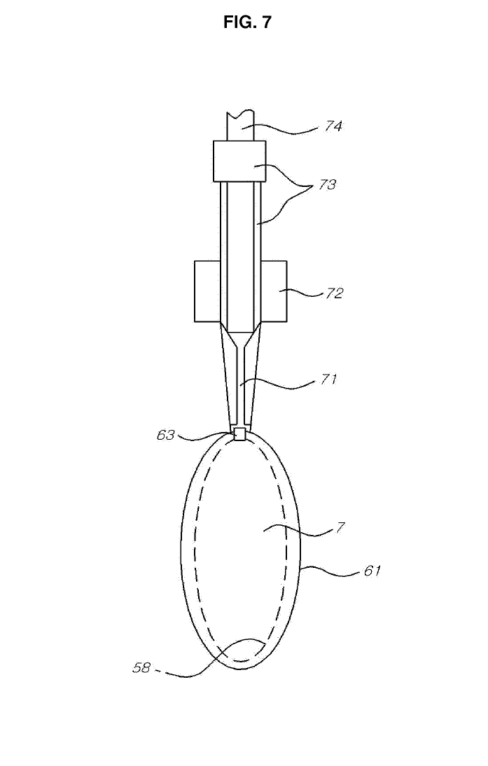

Here, as shown in FIG. 7, the grouting nozzle 66 used for the grouting process includes: a fused bonding rod injection part 71 for injecting a fused bonding rod of a solid phase into the damaged nozzle insertion hole 65 in contact with the nozzle insertion hole 65 so that the grouting nozzle 66 can penetrate into the nozzle insertion hole 65; a bonding rod heating part 72 having an electric heater which can heat to 200° C. and a thermal conductor; a bonding rod supporting part 73 having a cylindrical path for allowing the bonding rod of the solid phase to move toward the nozzle and a pusher which can push through the path; and the bonding rod 74 of the solid phase inserted into the bonding rod supporting part.

A material of the soft film for grouting is a strong cohesive bonding rod of the solid phase whose main ingredient is gelatin out of the soft film materials described in this specification, and is melted as much as it can penetrate the damage part even below 200° C.

Now, a method of injecting the soft film material will be described. A direction of the temporary vessel 61 is regulated in such a fashion that the damaged nozzle insertion hole 65 is located at the upper part, a welding rod of a solid phase 74 is inserted into the bonding rod supporting part of the solid phase 73, and then, the nozzle of the bonding rod injection part 71 gets in contact with the damaged nozzle insertion hole 65. After that, when the user pushes the electric heater of the bonding rod heating part 72 while pushing the nozzle toward the bonding rod injection part 71 using a push stick of the bonding rod supporting part 73, the front part of the welding rod 74 penetrates and is fused into the damaged nozzle insertion hole 65 through the bonding rod injection part 71 while being melted below 200° C., so that the nozzle insertion hole 57 is restored and sealed.

So, as shown in FIG. 5(f), the soft film gas-storing vessel 51 in which hydrogen gas and oil remainder are sealed is manufactured, and is perfectly prepared to be accommodated in the protective hard capsule.

In this embodiment, in order to charge hydrogen, as described in the third embodiment, the hydrogen gas charging system shown in FIG. 3(b) is used.

First, the temporary vessel for charging hydrogen gas and the temporary vessel in which oil is sealed are mounted on the hydrogen charging nozzle part and pumping oil separation valves 45 of n-number are closed. In a state where the hydrogen charging nozzle part is separated from the net-type hydrogen charging pipe 32, the pumping oil control valve 42 is closed and an oil discharge valve 44 is opened, so that an oil pump 41 is operated to discharge oil of an oil pump entrance line. After that, in the operation state of the oil pump, the oil discharge valve 44 is closed and the pumping oil control valve 42 is opened, so that oils contained in the net type pumping oil pipe 46, the hydrogen charging nozzles 64 of the hydrogen charging nozzle part 46 and the soft film gas-storing vessels 51 are pumped and discharged out. In this instance, it is necessary to adjust an amount of oil stuck on the vessel and an amount of oil stuck on the charging nozzle 64 so that the oil remainder 58 of 3% to 30% remains in the temporary vessel after charging of the hydrogen gas. Accordingly, when pressure of the pump entrance reaches a predetermined negative pressure in order to properly remain oil in the inside of the hydrogen charging nozzles 64 and their paths to some degree, the pumping oil control valve 42 is closed and the pumping oil separation valves 45 are opened, so that the hydrogen charging nozzle part communicates with the net-type hydrogen charging pipe 32. After that, the hydrogen gas control valve 35 of the hydrogen gas supply part is opened, and then, hydrogen gas is charged from the hydrogen gas supply source 33 through the hydrogen gas pressure controller 34 and the filter 36 till the hydrogen charging nozzles reach a predetermined pressure. When the charging of the hydrogen gas is finished, the hydrogen gas control valve 35 is closed to stop the supply of hydrogen gas, and the hydrogen gas charging nozzle is moved back to be removed from the temporary vessel. After that, the grouting nozzle 66 is connected to the nozzle insertion hole 65 of the temporary vessel, and then, the material of the soft film is bonded through the grouting process while the material of the soft film is injected into the damaged nozzle insertion hole 65 at predetermined injection pressure and temperature (below 200° C.). Thereby, the soft film gas-storing vessel 51 in which hydrogen gas and oil remainder are sealed is manufactured, and is completely ready to be accommodated inside the protective hard capsule.

### Embodiment 5

Now, hydrogen-generating sweets made by mixing a material, which generates hydrogen by colon bacteria flora, with sweets at the rate of 30% to 70% will be described.

Flatulence is a mixed gas generated during the digestion process of mammals as by-products, and is discharged out after being delivered to the rectum by the peristallic action of the digestive organs which make excreta lower from the large intestine, and the main ingredients of flatulence which is odorless and nontoxic are generally known as shown in the following Table 3.

TABLE 3

| | | | | Methane | |
|---|---|---|---|---|---|
| Item | Nitrogen | Oxygen | Hydrogen | Carbon | dioxide |
| Main | 20~90% | 0~10% | 0~30% | 10~30% | 0~10% |
| ingredient | | | | | |
| Average | 55% | 5% | 15% | 20% | 5% |
| Source of | During | During | Fermented | Fermented | Fermented |
| formation | suction of | suction of | in large | in large | in large |
| | air | air | intestine | intestine | intestine |
| | | | | | (30% of |
| | | | | | adults) |

Nitrogen and oxygen out of the flatulence which is odorless and nontoxic come from the air that we breathe, and oxygen is absorbed to the human body but nitrogen is not absorbed, and hence, is discharged out through flatulence. In the meantime, lots of hydrogen gas out of hydrogen gas, carbon dioxide and methane gas generated in the large intestine is delivered through the blood vessel, but most of useless carbon dioxide and methane gas are discharged out through flatulence.

Meanwhile, flatulence of bad smell contains skatole, indole, ammonia, and so on which are caused by animal protein, and sulfur-containing compound, such as methaneethiol, hydrogen sulfide, dimethylsulfide, and so on, and is about 1% of gases generated in the large intestine.

Human beings emits gas of 500 cm³ to 700 cm³ per a day about fifteen times based on adults, and the generated hydrogen gas is 75 cm³ to 105 cm³ a day, and some of the generated hydrogen gas is discharged out through the anus and the other hydrogen gas is introduced into the blood stream of the intestinal wall by the diffusion process through the lumen, which is a path for passing nutrients in the intestine when it is higher than partial pressure in the blood. The hydrogen gas delivered along the blood stream penetrates into cells, some of the hydrogen gas comes out from the blood when the blood passes through the lung and is discharged together with exhalation. In this instance, most of carbon dioxide and methane gas generated together with hydrogen gas are discharged through the anus, and some of the carbon dioxide and methane gas enter the blood stream of the intestinal wall and mixed with other carbon dioxide of varicose vein.

There are many studies for supporting that hydrogen is generated by being fermented by colon bacteria flora and the generated hydrogen provides a strong antioxidation:

—Breath hydrogen testing as a physiology laboratory exercise for medical students, Ramon G. Montes, Richard F. Gottal, School of Medicine, Johns Hopkins University, accepted Dec. 23, 1991—It is a method of establishing absorption and decrease of carbohydrate in the breath hydrogen testing. Breath hydrogen testing (BHT) was carried out as a studying tool, a sample obtained at the time of exhalation through the nasal prong technology is analyzed by gas chromatography. Finally, students who ate lactose, fructose and lactulose showed more hydrogen gas due to malabsorption in the small intestine than students who ate glucose or sucrose, which is a monosaccaride, and it is caused by lack of lactase, which is an enzyme.

The result of BHT is influenced by elements related with quantitative and qualitative aspects of the colon bacteria flora. An object of the study is to find whether or not hydrogen gas discharge capacity is changed after a patient orally took lactulose in the case that there is a loss in the original state of the colon—Influence of colectomy on hydrogen excretion in breath, Francesc Casellas, Francesc Casellas, A. Torrejon, accepted Oct. 14, 2008—As a result of the test, patients who entirely removed their small intestines were still lower in discharge amount of hydrogen gas than patients who were in good management of the colon or partially removed the colon.

In the test, female students took a-D galactosidase, which is an enzyme to hydrolyze raffinose, and their biorhythm patterns were traced, and for the test, BHT was used. —Circadian rhythm of breath hydrogen in young women, Mieko Kagaya, Mayumi Iwata, accepted Jan. 23, 1998—In the report, as a result of the test, the case that they took a-D galactosidase (17 ppm) was 7 ppm lower than the case that they did not took a-D galactosidase (24 ppm), and more hydrogen gas was generated when they took lots of dietary fiber. Through the above, it is assumed that more hydrogen gas are generated when female students take lots of dietary fiber containing oligosaccharides.

After 8 healthy persons took curry containing turmeric, which is a raw material of curcumin, and also took turmeric-free curry, and obtained samples were analyzed by gas chromatography. —Effect on dietary turmeric on breath hydrogen, Akito Shimouchi, Kazutoshi Nose, Dig Dis Sci, 2009; 54(8): 1725˜1729—As a result, turmeric-containing curry remarkably increased breath hydrogen in comparison with turmeric-free curry, reduced the passing time of the small intestine, and activated motility of the intestine and fermentation of carbohydrate in the colon.

—Antioxidant and anti-inflammatory properties of curcumin, Venugopal P, Menon and Adluri Ram Sudheer, I.E. Magazine, September 2001—Cylooxygenase-2, Lypooxygenase of curcumin and nitric oxide synthase, which is derivatives, are enzymes arbitrating the inflammation process. Inflammation is closely related with promotion of tumor, and hence, has chemically preventive properties to carcinogenesis, and a damage of peroxidation of the lipid membrane to which active oxygen intervenes and the oxidative damages of DNA and protein are related with chronic pathological complications, and hence, it is assumed that they are related with intestinal hydrogen generation of curcumin.

Curcumin protects the skin, the mouth, the small intestine, the colon from carcinogenesis, and have showed through tumor models of various animals that curcumin restrains angiogenesis and transition that new blood vessel is formed from the existing blood vessel. —Cancer chemopreventive effects of curcumin, Young-Joon Surh, Kyung-Soo Chun, Advances in Experimental Medicine and Biology, vol 595—curcumin restrains spread of cancer cells by collecting cancer cells and inducing death of programmed cells in various stages of the cell cycle. Moreover, curcumin restrains biological activation of carcinogenesis by restraining cytochrome P450 isozyme which makes chemicals nontoxic by the electron delivery chain, and hence, it is assumed that it is also associated with intestinal hydrogen generation of curcumin.

There was a study to check synergy effects of dietary curcumin, capsaicin and spices, which are bioactive compounds, through induced hypercholesterolemic rats in relation with hypolipidemic and antioxidant effects helpful to health. —Hypolipidemic and antioxidant effects of dietary curcumin and capsaicin in induced hypercholesterolemic rats, H. Manjunatha and K. Srinivasan, published on line, October 2007—Curcumin and capsaicin lowered lipoperoxide in the liver and the blood in the induced hypercholesterolemic rats, reinforced ascorbic acid of the liver in normal rats and reinforced glutathione, which is a reducing agent, in the induced hypercholesterolemic rats, and hence, it is assumed that it is associated with the intestinal hydrogen gas by curcumin and/or capsaicin.

As materials promoting generation of nontoxic and odorless gas including hydrogen gas by being prey to colon bacteria flora existing in the colon, which is a part of the large intestine, and being fermented without absorption by hydrolysis in the small intestine due to enzymes which do not exist in human's digestive organs or lacking enzymes,

First, there are lactose and fructose which are contained in milk products and are oligosaccharides of carbohydrate, and lactulose which is artificially synthesized. When people are grown up, the above materials are not decomposed in the small intestine due to lack of lactase which is an enzyme hydrolyzing the above materials.

Second, there are raffinose which is trisaccaride, stachyose which is tetrasaccaride, verbascose which is pentasaccaride in the RFO group, which are contained in beans, plants, cabbages, broccolis, asparagus, and so on, and which are oligosaccharides of carbohydrate. They are not decomposed and absorbed in the small intestine because there is no a-galactosidase (a-GAL), which is an enzyme hydrolyzing the above materials, in the digestive organs.

Third, there are curcumin of turmeric used as dietary spice, capsaicin belonging to acid amid group of peppers, inulin which is polysaccharide contained in artichokes, burdocks, and so on, gingerrol of gingers, allicin of sulfur compounds contained in garlics. They are not decomposed and absorbed in the small intestine because there is no enzyme hydrolyzing the above materials in the digestive organs.

Fourth, there are gluten contained in endosperms of wheat, rye, barley, oats, and so on consists of gliadin and glutenin, which are protein. Glutenin has sulfur atoms associated with netlike interconnection, and hence, is not decomposed and absorbed in the small intestine because there is no enzyme hydrolyzing gluten.

As a sweet producing method, in the second embodiment, in the step of making the massecuites from which some moisture is removed, in the step of adding the compounds inducing that sweet vessels or sweets generate hydrogen gas useful in the intestine, intestinal hydrogen generating sweets are produced through the steps of adding at least one compounds selected from lactose extracted from milk products and lactulose obtained by artificially synthesizing fructose and galactose, adding at least one compounds selected from raffinose which is trisaccaride, stachyose which is tetrasaccaride, verbascose which is pentasaccaride in the RFO group, which are contained in beans, plants, cabbages, broccolis, asparagus, kali, radishes, napa cabbages, and so on, adding at least one compounds selected from curcumin extracted from turmeric used as dietary spice, capsaicin of peppers, inulin extracted from artichokes, burdocks, and so on, gingerrol extracted from gingers, allicin extracted from garlics, or adding at least one compounds selected from gluten and glutenin extracted from wheat, rye, barley, oats, and so on, and cooling the massecuites for molding and jetting.

After the ingredients of the massecuites are first adjusted, in the step of adding the compounds inducing that sweet vessels or sweets generate hydrogen gas useful in the intestine, in order to mold or jet the massecuites by adding at least one compounds selected from lactose, lactulose, raffinose, stachyose, verbascose, curcumin, capsaicin, inulin, gingerrol, allicin, gluten, glutenin, and so on, the intestinal hydrogen generating sweets are produced by cooling the massecuites.

In the massecuites made from the compounds of sugar-alcohol, in order to promote generation of hydrogen gas in the intestine, in order to remove the function of storing hydrogen gas therein and add only the compounds inducing generation of more hydrogen gas useful in the intestine, in order to mold or jet the massecuites by adding at least one compounds selected from lactose, lactulose, raffinose, stachyose, verbascose, curcumin, capsaicin, inulin, gingerrol, allicin, gluten, glutenin, and so on, the intestinal hydrogen generating sweets are produced by cooling the massecuites. In this instance, there is no special limitation in shape and size of the sweets.

As an ideal method of measuring useful gas additionally generated in the intestine by taking the intestinal hydrogen generating sweets, the breath hydrogen test (BHT) is conventionally used. After a patient takes a proper amount necessary for the test, a plastic syringe inhales a sufficient amount necessary for analysis of hydrogen gas every exhalation using the nasal prong system within 12 hours after intake. Analysis of hydrogen gas contained in the sample is carried out by gas chromatography for semiconductors. In the BHT, normal adults are 6 ppm to 14 ppm in the level of breath hydrogen, an increase amount of hydrogen gas when the patient takes the intestinal hydrogen generating sweets is 2 ppm to 10 ppm by adding hydrogen contained in the vessel, and preferably, the ingredients of the massecuites or the hydrogen gas containing amount of the vessel is controlled such that the increase amount becomes 2 ppm.

### Embodiment 6

In order to dissolve the gas-storing vessel 2 in the intestine of the human body after a predetermined period of time after the patient takes the gas-storing vessel 2 with drinking water, under the nitrogen gas atmosphere, the gas-storing vessel 2 is put in the lower part 3 of the conventional hard capsule and the upper part 4 of the hard capsule is covered. After that, the bonding part 5 of the hard capsule where the lower part 3 and the upper part 4 of the hard capsule are overlapped is bonded, so that the hydrogen-storing vessel 1 is completely manufactured.

The hard capsule 16 is manufactured according to the conventional manufacturing method, but an area of the bonding part where the upper part and the lower part of the hard capsule are overlapped is maximized as shown in FIG. 1 in order to enhance sealability, tolerance to humidity and tensile strength in comparison with the conventional hard capsules containing granulation or powder, and as shown in FIG. 1, the bonding part is located at the center of the hard capsule 16 in order to be protected from external shock.

The hard capsule may be made of water-soluble cellulose inductor and gelant, or made of gelatin, polyethylene glycol (PEG), glycerin, and so on, or nano silver coating may be added to the hard capsule. The hard capsule is high in viscosity at high temperature, and must be dissolved in refined water heated at 37° C. within 15 minutes.

The soft film gas-storing vessel may be packed as it is, but it is good to pack it once more in order to prevent a damage due to carelessness during delivery and to be dissolved in the intestine of the human body after a lapse of a predetermined period of time after a patient takes the vessel together with drinking water. So, under the nitrogen gas atmosphere, the soft film gas-storing vessel in which hydrogen gas is charged is put in the lower part of the conventional hard capsule and is covered with the upper part of the hard capsule, and then, the bonding part of the hard capsule where the upper part and the lower part of the hard capsule are overlapped is bonded, whereby the hydrogen-storing vessel is completely manufactured.

In selection of packing methods of the hydrogen-storing vessel 1, in consideration of a long-term storage and safety from fire, in the conventional pocket type drug packing structure, based on an aluminum layer, a nylon layer and one of high-density polyethylene (HDPE), terephthalate (PET) and polyvinyl chloride (PVC) are laid one on top of the other and thermally punched so that an upper film having an open dome part is formed. After that, the hydrogen-storing vessel 1 is put in the dome part and an aluminum foil layer (press through pack foil) which is a lower film comes in contact with the vessel. Thermosetting and sealable materials is selected out of vinyl resin, polyurethane resin and thermosetting adhesive resin, and in order to enhance block effects of humidity and sunlight, a dry air layer may be added between the aluminum layer, which is the upper film, and the HDPE.

In order to store and deliver the packing structures of the hydrogen-storing vessels for a long time, it is more preferable to use aluminum packs, which is small-sized, compressed, sealed and insulated.

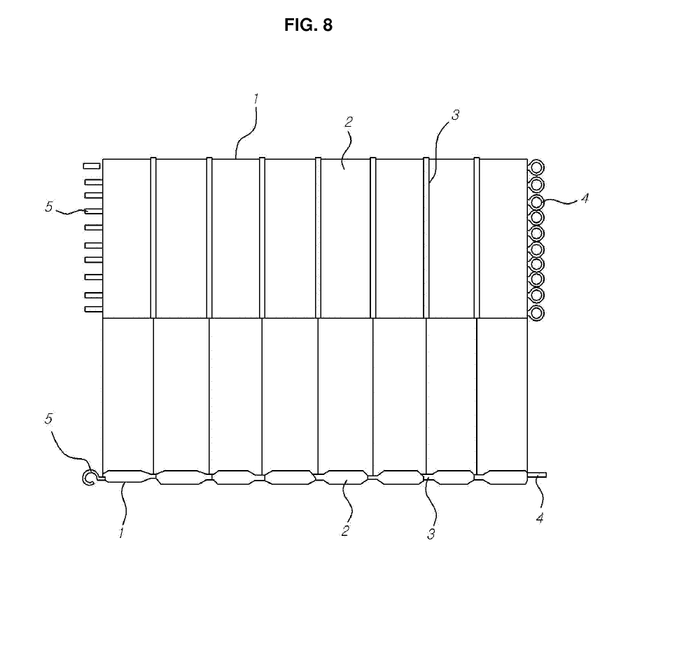

FIG. 8 is an exemplary view of a water jacket for fire resistance according to a preferred embodiment of the present invention, wherein the upper diagram is a plan view of the water jacket and the lower diagram is a side view of the water jacket.

For safe storage and delivery of the sweet vessels storing hydrogen gas and products packing the vessels, as shown in FIG. 8, a unit water jacket 1 of a cushion form is manufactured of at least one compounds selected from polypropylene, low-density polypropylene, and high-density polypropylene which can be melded at temperature of 120° C. Concave portions 3 and convex portions 2 are arranged in turn, so that the water jacket can contain water or fire-resistant antifreeze. The unit water jacket 1 include a female hook 4 made of plastic or metal and arranged at one end of a longer side thereof and a male hook 5 arranged at the other end.

When the hydrogen-generating sweet vessel is packed, two pieces of the water jackets are rolled on the vessel in vertical and horizontal directions, namely, one is rolled vertically and the other one is rolled horizontally. After the water jackets are rolled on the vessel, the hydrogen-generating sweet vessels are put and packed in a box.

In other words, all six sides of the packed products are wrapped with the water jackets, and then, put in a storage box or a carrying box, so that the packing method provides fire-preventing function and cooling function at high temperature, and the packed products can be connected with each other using the hooks formed at both sides of the unit water jackets according to the dimensions of the carrying box.

### Embodiment 7

In the cylindrical gas-storing vessel of the capsule type made of the material which is water-soluble, besides hydrogen gas, carbon monoxide, nitric oxide, Xenon, helium, and others can be charged, and when they are supplied to the human body, they can cause various medical effects.

As medical gas molecules providing medical solutions, there are oxygen (O₂), nitrous oxide (N₂O), which are typically known medical gas molecules, and according to development of medical technology, recently, lots of reports on roles of carbon monoxide (CO), nitric oxide (NO), Xenon (Xe), and helium (He) have been published.

Effects of molecular gasses on the human body will be summarized as follows. Hydrogen, as described above, provides antioxidation to protect tissues by preventing and curing a damage of cells by oxidation in the human body or in the body of mammal, and particularly, shows a great effect in removing hydroxyl radical (*OH) and peroxynitrite (ONOO—), which are strongly active oxygen generated in each part of the human body.

Carbon monoxide is a toxic gas causing hypoxia of cell tissues by deteriorating oxygen delivery capacity of blood at high density, but can prevent an oxidative damage of cells by hypoxia (ischaemia), such as ischemia reperfusion injury, through antioxidation at low density (ppm unit), and can remove superoxide radical (O₂⁻) generated in mitochondria which synthesizes energies in the human body, and can enhance effects of combination therapies together with hydrogen gas.

Nitric oxide increases blood supply in the blood vessel as a signal delivery molecule in the human body at low density and protect tissues from an oxidative injury of cells, but needs to keep balance because it increases peroxynitrite, which is active oxygen, at high density.

Xenon and helium are inert, and can prevent ischemia reperfusion injury of the heart muscle because it can protect cells from oxidation by helping activation of signal at low concentration in the human body.

Such molecular gases are sealed in gas-storing vessels, and can be supplied to the human body when users take them. The gas-storing vessel, the hydrogen-charging method, and the hydrogen-storing vessel manufacturing method can be achieved by applying the technologies described above in order to seal hydrogen gas.

1. A gas-storing vessel of a cylindrical capsule type made of a material, which can store gas and is dissolved in water so that gas leaks out.

2. The gas-storing vessel according to claim 1, wherein the vessel is made with massecuites, which are at least one compounds selected from saccharide, such as sugar, maltose, grain syrup and others, which are ingredients of soft sweets, amorphous hard sweets, and sugarfree hard sweets, carbohydrate syrup compounds of starch, maltitol, mannitol, sorbitol, Isomalt, xylitol and others, and compounds of polyols, or
at least one additive selected from gelatin, glycerin, ferrous fumarate, vitamin C, vitamin E, polyphenol, carotene, calcium, and others is added to the massecuites.

3. The gas-storing vessel according to claim 1, wherein a hard capsule which is larger and thinner than the cylindrical capsule type vessel is mounted on the outer face of the cylindrical capsule type vessel in order to store hydrogen.

4. A method of manufacturing the gas-storing vessel according to claim 1, the method comprising the steps of:
molding a temporary vessel made of a material melted by heat and surrounding a hydrogen charging nozzle having a volume as big as hydrogen can be charged therein;
mounting a thermocompression band at a portion excepting the length corresponding to the hydrogen charging space, removing air contained in the temporary vessel using the hydrogen charging nozzle, and charging hydrogen below the atmospheric pressure; and
retreating the hydrogen charging nozzle to the end of the thermocompression band and sealing an entrance of the temporary vessel by heating the thermocompression band and tightening the band.

5. The method of manufacturing the gas-storing vessel according to claim 4, wherein in the temporary vessel molding step, the vessel is made with massecuites, which are at least one compounds selected from saccharide, such as sugar, maltose, grain syrup and others, which are ingredients of soft sweets, amorphous hard sweets, and sugarfree hard sweets,
carbohydrate syrup compounds of starch, maltitol, mannitol, sorbitol, Isomalt, xylitol and others, and compounds of polyols, and
wherein the massecuites are made by adding at least one compounds selected from gelatin, glycerin, ferrous fumarate, vitamin C, vitamin E, polyphenol, carotene, calcium, and others, and is molded in a casting mold.

6. The method of manufacturing the gas-storing vessel according to claim 4, wherein in the temporary vessel molding step, the vessel is made of a material, which has good airtightness, is low in calorific value, is thermally deformable when it is compressed by heat, and has glass transition temperature of more than 38° C. with water contents of more than 3%, and
wherein purity of hydrogen gas charged in the vessel is more than 99.999%.

7. The gas-storing vessel according to claim 1, wherein the vessel of the cylindrical capsule is water-soluble, but has a soft film which is not dissolved in organic solvent.

8. The gas-storing vessel according to claim 7, wherein the soft film is made of at least one compounds selected from general gelatin, succinic acid gelatin, glycerin, starch, and as plasticizer or softner, sorbitol, sorbitan or at least one compound is selected from sorbite group, polyglycytol syrups, sucrose, mannitol, xylitol, maltose, reduced maltose syrup, maltitol, polyethylene glycol, and
wherein the substrate of the soft film is at least one selected from refined water, black ferrous fumarate, red ferrous fumarate, and so on.

9. The gas-storing vessel according to claim 7, wherein hydrogen gas in the vessel is 70% to 97% in volume, and at least one ingredient selected from soybean oil, safflower oil, purified fish oil, purified processed oil, sesame oil, red-pepper seed oil, wheat germ oil, grape seed oil, olive oil, rape seed oil, evening primrose oil, and fruit flavor oil is 3% to 30% in volume.

10. The gas-storing vessel according to claim 7, wherein the gas is hydrogen, the vessel is molded in consideration of pressure change in atmospheric pressure, thickness of a cooled and dried film is 0.7 mm to 1.0 mm, thickness of the joint part is 0.6 mm to 0.95 mm, and thickness of the restored nozzle insertion hole is 0.7 mm to 1.0 mm.

11. A method of manufacturing the soft film gas-storing vessel of claim 7, the method comprising the steps of:
making a soft film temporary vessel by putting and sealing oil into a soft film,
inserting a hydrogen charging nozzle into the temporary vessel, removing oil contained in the temporary vessel, and injecting hydrogen gas; and
removing the hydrogen charging nozzle from the vessel and sealing the nozzle insertion hole, which is perforated in the temporary vessel by the hydrogen charging nozzle, with a grouting material.

12. The method of charging and sealing hydrogen gas according to claim 11, wherein the hydrogen charging nozzle has a sharp end like a needle, and oil of 3% to 30% is remained in the temporary vessel when the oil is removed from the vessel.

13. The gas-storing vessel according to claim 1, wherein the cylindrical capsule type vessel comprises:
a sweet vessel made by mixing a material, which generates hydrogen gas in the large intestine by colon bacteria flora, with sweets at the ratio of (30˜70):(70˜30), and
hydrogen charged in the sweet vessel.

14. The hydrogen generating material according to claim 13, made of at least one selected from lactose, lactulose, raffinose, stachyose, verbascose, curcumin, capsaicin, inulin, gingerrol, allicin, gluten, glutenin, and so on.

15. The gas-storing vessel according to claim 1, wherein one selected from hydrogen gas, carbon monoxide, nitric oxide, Xenon, and helium, which are medical gases, is stored in the cylindrical capsule type vessel.

16. A method of manufacturing the gas-storing vessel according to claim 2, the method comprising the steps of:
molding a temporary vessel made of a material melted by heat and surrounding a hydrogen charging nozzle having a volume as big as hydrogen can be charged therein;
mounting a thermocompression band at a portion excepting the length corresponding to the hydrogen charging space, removing air contained in the temporary vessel using the hydrogen charging nozzle, and charging hydrogen below the atmospheric pressure; and
retreating the hydrogen charging nozzle to the end of the thermocompression band and sealing an entrance of the temporary vessel by heating the thermocompression band and tightening the band.

17. The method of manufacturing the gas-storing vessel according to claim 16, wherein in the temporary vessel molding step, the vessel is made with massecuites, which are at least one compounds selected from saccharide, such as sugar, maltose, grain syrup and others, which are ingredients of soft sweets, amorphous hard sweets, and sugarfree hard sweets,
carbohydrate syrup compounds of starch, maltitol, mannitol, sorbitol, Isomalt, xylitol and others, and compounds of polyols, and
wherein the massecuites are made by adding at least one compounds selected from gelatin, glycerin, ferrous fumarate, vitamin C, vitamin E, polyphenol, carotene, calcium, and others, and is molded in a casting mold.

18. The method of manufacturing the gas-storing vessel according to claim 16, wherein in the temporary vessel molding step, the vessel is made of a material, which has good airtightness, is low in calorific value, is thermally deformable when it is compressed by heat, and has glass transition temperature of more than 38° C. with water contents of more than 3%, and
wherein purity of hydrogen gas charged in the vessel is more than 99.999%.
